(19) United States
(12) Patent Application Publication
(10) Pub. No.: US 2021/0059983 A1
(43) Pub. Date: Mar. 4, 2021

(54) METHODS AND MATERIALS FOR IDENTIFYING AND TREATING CANCER

(71) Applicants: Amit K. Verma, Bronx, NY (US); Mayo Foundation for Medical Education and Research, Rochester, MN (US)

(72) Inventors: Niraj K. Shenoy, Rochester, MN (US); Thomas E. Witzig, Rochester, MN (US); Amit K. Verma, Bronx, NY (US)

(21) Appl. No.: 16/963,062

(22) PCT Filed: May 17, 2019
(86) PCT No.: PCT/US2019/032883

§ 371 Date: Jul. 17, 2020

Related U.S. Application Data
(60) Provisional application No. 62/673,582, filed on May 18, 2018.

(51) Int. Cl. A61K 31/375; A61P 35/00

(52) U.S. Cl. CPC A61K 31/375; A61P 35/00

(57) ABSTRACT

This document provides methods and materials involved in identifying and treating mammals having a cancer (e.g., a clear cell renal cell carcinoma (ccRCC)) based, at least in part on, 5-hydroxymethylcytosine (5hmC) levels. For example, methods and materials for administering a high dose of ascorbic acid (AA) with or without an additional chemotherapeutic agent to a mammal identified as having cancer having a reduced level of 5hmC are provided. Methods and materials for administering a chemotherapeutic agent without administering a high dose of AA to a mammal identified as having cancer exhibiting 5hmC expression also are provided.

### CROSS-REFERENCE TO RELATED APPLICATIONS

This application claims the benefit of U.S. Patent Application Ser. No. 62/673,582, filed on May 18, 2018. The disclosure of the prior application is considered part of (and is incorporated by reference in) the disclosure of this application.

### STATEMENT AS TO FEDERALLY SPONSORED RESEARCH

This invention was made with government support under CA087274 awarded by National Institutes of Health. The government has certain rights in the invention.

### BACKGROUND

### 1. Technical Field

This document relates to methods and materials involved in identifying and treating mammals having a cancer (e.g., a clear cell renal cell carcinoma (ccRCC)) based, at least in part on, 5-hydroxymethylcytosine (5hmC) levels in the cancer cells. For example, this document provides methods and materials for administering a high dose of ascorbic acid (AA) with or without an additional chemotherapeutic agent or a targeted therapy agent to a mammal identified as having cancer having a reduced level of 5hmC. This document also provides methods and materials for administering a chemotherapeutic agent without administering a high dose of AA to a mammal identified as having cancer exhibiting a high level of 5hmC expression.

### 2. Background Information

Metastatic renal cell cancer is a generally incurable malignancy that needs new treatments based on molecular insights. ccRCC is an epigenetically aberrant solid tumor, characterized by widespread DNA cytosine hypermethylation (Hu et al., Clin. Cancer Res., 20(16):4349-60 (2014); Cancer Genome Atlas Research Network, Nature, 499(7456):43-9 (2013); Shenoy et al., J. Hematol. Oncol., 8:88 (2015); and Shenoy et al., Ann. Oncol., 27(9):1685-95 (2016)). Aberrant methylation was particularly enriched in kidney-specific enhancer regions (H3K4Mel positive regions) associated with silencing of genes such as the TGF-beta regulator SMAD6. Similar findings also were seen in the recent TCGA analysis of ccRCC (Cancer Genome Atlas Research Network, Nature, 499(7456):43-9 (2013)).

### SUMMARY

This document provides methods and materials involved in identifying and treating mammals having a cancer (e.g., a ccRCC) based, at least in part on, 5hmC levels. For example, this document provides methods and materials for administering a high dose of AA with or without an additional chemotherapeutic agent or a targeted therapy agent to a mammal identified as having cancer having a reduced level or intensity of intratumoral 5hmC (e.g., absent, low, mild, or moderate 5hmC levels where less than 90 percent of cancer cells are positive for 5hmC or 5hmC expression). Having the ability to identify a mammal having cancer exhibiting a reduced level or intensity of intratumoral 5hmC as described herein can allow clinicians and patients to proceed with the use of a high dose AA to treat that cancer in an effective manner. Such a treatment can be a high dose AA alone treatment or in combination high dose AA with one or more chemotherapeutic agents or targeted therapy agents. In the setting of surgically resected localized cancer with reduced 5hmC, treatment options would be ascorbic acid and/or a chemotherapeutic agent and/or a targeted therapy agent.

This document also provides methods and materials for avoiding the use of chemotherapeutic agents or targeted therapies or high dose AA in a mammal identified as having localized cancer exhibiting high 5hmC expression (e.g., “marked intensity”, correlating with greater than 90 percent of cancer cells are positive for 5hmC or 5hmC expression), after resection of localized disease. Having the ability to identify a mammal having cancer exhibiting 5hmC expression (e.g., greater than 90 percent of cancer cells are positive for 5hmC or 5hmC expression) as described herein can allow clinicians and patients to avoid the unnecessary use of a high dose AA in the adjuvant setting (e.g., after surgical resection of localized cancer), to avoid the unnecessary use of chemotherapeutic agents, and/or to avoid the unnecessary use of targeted therapies. The reason is that mammals with cancers having “marked” expression have excellent prognosis after complete resection of localized disease and have a very low chance of disease recurrence (see, e.g., FIG. 2F).

In general, one aspect of this document features a method for identifying a mammal as having a cancer comprising a reduced level of 5hmC. The method comprises (or consists essentially of or consists of) (a) determining that less than 90 percent of cancer cells of the mammal are positive for the presence of 5hmC, and (b) classifying the mammal as having the cancer. The mammal can be a human. The cancer can be a kidney cancer. The presence of 5hmC can be determined using an anti-5hmC antibody.

In another aspect, this document features a method for identifying a mammal as having a cancer without a reduced level of 5hmC. The method comprises (or consists essentially of or consists of) (a) determining that greater than 90 percent of cancer cells of the mammal are positive for the presence of 5hmC, and (b) classifying the mammal as having the cancer. The mammal can be a human. The cancer can be a kidney cancer. The presence of 5hmC can be determined using an anti-5hmC antibody.

In another aspect, this document features a method for treating cancer. The method comprises (or consists essentially of or consists of) (a) identifying a mammal as having a cancer comprising a reduced level of 5hmC, and (b) administering a high dose of AA, a chemotherapeutic agent, or a targeted therapy to the mammal. The mammal can be a human. The cancer can be a kidney cancer. The cancer can be a ccRCC. The identifying step can comprise determining that less than 90 percent of cancer cells of the mammal are positive for the presence of 5hmC. The presence of 5hmC can be determined using an anti-5hmC antibody. The method can comprise administering the high dose of AA to the mammal. The high dose of AA can be administered as the sole active ingredient against the cancer. The method can comprise administering the chemotherapeutic agent to the mammal. The chemotherapeutic agent can be cisplatin, carboplatin, gemcitabine, etoposide, temozolamide, paclitaxel, 5-FU, or oxaliplatin. The chemotherapeutic agent can be administered as the sole active ingredient against the cancer. The high dose of AA and the chemotherapeutic agent can be administered to the mammal. The chemotherapeutic agent and the high dose of AA can be administered during the same day. The chemotherapeutic agent can be administered before the high dose of AA. The chemotherapeutic agent can be administered after the high dose of AA.

In another aspect, this document features a method for treating metastatic cancer or cancer in an unresectable setting. The method comprises (or consists essentially of or consists of) (a) identifying a mammal as having a cancer comprising a reduced level of 5hmC, and (b) administering, to the mammal, a high dose of AA in combination with one or more chemotherapeutic agents or one or more targeted therapies. The mammal can be a human. The cancer can be a kidney cancer. The cancer can be a ccRCC. The identifying step can comprise determining that less than 90 percent of cancer cells of the mammal are positive for the presence of 5hmC. The presence of 5hmC can be determined using an anti-5hmC antibody.

In another aspect, this document features a method for treating metastatic cancer or cancer in an unresectable setting. The method comprises (or consists essentially of or consists of) administering, to a mammal identified as having a cancer comprising a reduced level of 5hmC, a high dose of AA in combination with one or more chemotherapeutic agents or one or more targeted therapies. The mammal can be a human. The cancer can be a kidney cancer. The cancer can be a ccRCC.

In another aspect, this document features a method for treating cancer in a localized setting. The method comprises (or consists essentially of or consists of) (a) identifying a mammal as having a cancer comprising a reduced level of 5hmC, and (b) administering, to the mammal, a high dose of AA, one or more chemotherapeutic agents, or one or more targeted therapies. The mammal can be a human. The cancer can be a kidney cancer. The cancer can be a ccRCC. The identifying step can comprise determining that less than 90 percent of cancer cells of the mammal are positive for the presence of 5hmC. The presence of 5hmC can be determined using an anti-5hmC antibody.

In another aspect, this document features a method for treating cancer in a localized setting. The method comprises (or consists essentially of or consists of) administering, to a mammal identified as having a cancer comprising a reduced level of 5hmC, a high dose of AA, one or more chemotherapeutic agents, or one or more targeted therapies. The mammal can be a human. The cancer can be a kidney cancer. The cancer can be a ccRCC.

In another aspect, this document features a method for treating cancer in a manner that avoids an unnecessary administration of a high dose of AA, a chemotherapeutic agent, and a targeted therapy. The method comprises (or consists essentially of or consists of) (a) identifying a mammal as having a cancer lacking a reduced level of 5hmC, and (b) monitoring the mammal without administering a high dose of AA to the mammal, without administering a chemotherapeutic agent to the mammal, and without administering a targeted therapy to the mammal. The mammal can be a human. The cancer can be a kidney cancer. The cancer can be a ccRCC. The identifying step can comprise determining that greater than 90 percent of cancer cells of the mammal are positive for the presence of 5hmC. The presence of 5hmC can be determined using an anti-5hmC antibody.

In another aspect, this document features a method for treating cancer in a manner that avoids an unnecessary administration of a high dose of AA, a chemotherapeutic agent, and a targeted therapy. The method comprises (or consists essentially of or consists of) monitoring a mammal identified as having a cancer lacking a reduced level of 5hmC without administering a high dose of AA to the mammal, without administering a chemotherapeutic agent to the mammal, and without administering a targeted therapy to the mammal. The mammal can be a human. The cancer can be a kidney cancer. The cancer can be a ccRCC.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A-1I. Loss of 5hmC is strongly associated with features of tumor aggressiveness in clear cell RCC. A: Correlation between median percent positive 5hmC and 5hmC intensity in IHC (p<0.001). B: Higher grade ccRCC is associated with loss of 5hmC (p<0.001). C: Representative pictures of low grade and high grade ccRCC with 5hmC IHC. D: Loss of 5hmC correlates with higher SSIGN score (which predicts increased risk of progression of ccRCC after nephrectomy) (p<0.001). E: Increased tumor size in ccRCC is associated with loss of 5hmC (p<0.001). F: Nodal metastasis in ccRCC is associated with loss of 5hmC (p<0.001). G: Presence of systemic metastatic disease in ccRCC is associated with loss of 5hmC (p<0.001). H: Presence of coagulative tumor necrosis is associated with loss of 5hmC (p<0.001). I: Presence of sarcomatoid differentiation is associated with loss of 5hmC (p<0.001).

FIGS. 2A-2F. Loss of 5hmC is an independent prognostic factor in clear cell RCC and predicts shortened time to metastatic disease after surgical resection for M0 disease. A: Univariable association of percent positive 5hmC with death from any cause (HR: 0.82, 95% CI: 0.79-0.85, p<0.001, n=568 patients). B: Univariable association of percent positive 5hmC with death from RCC (HR: 0.74, 95% CI: 0.70-0.78, p<0.001, n=568 patients). C: Univariable association of 5hmC intensity with overall survival (median OS in the absent, mild, and moderate 5hmC intensity cohorts occurred at 2.4, 4.1, and 10.5 years, respectively. Median OS in the marked group has not been reached). D: Univariable association of 5hmC intensity with cancer specific survival (CSS) (median CSS in the absent and mild intensity cohorts occurred at 2.7 and 6.8 years, respectively. Median CSS in the moderate and marked 5hmC intensity group has not been reached. 10-year CSS in the marked 5hmC intensity group is 90%). E: Univariable association of percent positive 5hmC with progression following surgery for M0 disease (HR: 0.76, 95% CI: 0.72-0.80, p<0.001). F: Univariable association of 5hmC intensity with progression-free survival among M0 patients (median PFS in the absent and mild intensity cohort occurred at 0.8 and 4.3 years, respectively. Median CSS in the moderate and marked 5hmC intensity group has not been reached. 10-year PFS in the marked 5hmC intensity group is 81%).

FIG. 3A-3I. L-2-hydroxyglutarate dehydrogenase (L2HGDH) deletions and underexpression are seen in ccRCC and significantly associated with adverse prognosis. A: Analysis of TCGA dataset revealed that TET-2 is mutated (heterozygous) only in 2.2% of ccRCC tumors. The mutations seen are predominantly missense (green) or non-sense (black) and are predicted to result in truncated proteins. B: TCGA analysis revealed no significant difference in TET-2 expression between ccRCC and normal kidney. C: TET-2 immunohistochemistry revealed no difference in expression patterns between high grade and low grade ccRCC. A, B and C, taken together, indicates that neither the mutation rate nor the expression of TET-2/TET-1 explains the loss of 5hmC in higher grade ccRCC. D: L2HGDH gene expression comparison between matched normal kidney and ccRCC tumor from TCGA (n=72). The mean normalized log 2 expression for the normal group is 9.28 and that of the tumor group is 7.75 (paired t test, p<2.2e-16). E: Copy number variation data for 528 patients (TCGA) with ccRCC-deletion: 217 (41%), no CNV: 295 (56%), amplification: 16 (3%). F: TCGA analysis of methylation status of ccRCC tumors based on low L2HGDH and high L2HGDH. G: L2HGDH IHC (n=40; 20 with high 5hmC and 20 with low 5hmC) suggests that loss of L2HGDH is associated with loss of 5hmC (p=0.009). H: Representative pictures showing loss of L2HDH in lower 5hmC ccRCC. I: Lower L2HGDH expression is associated with shorter survival (p<0.0001). n=533 ccRCC patients, grouped based on L2HGDH expression <= or > than median. Survival analysis performed with log rank test. The median survival (in days) for the low L2HGDH group is 1980. Median not reached for the high L2HGDH group.

FIG. 4A-4H. AA leads to increased TET activity and 5hmC levels in RCC cells. A: Schematic showing the role of Ascorbic acid as an essential cofactor for TET enzymatic activity. B: Intracellular L-2HG levels measured by mass spectrometry in ccRCC cell line 7860 is much higher than the immortalized normal kidney cell line (HKC8) (Ttest, *P val<0.05, N=2). C, D: TET activity was measured in vitro with AA treated RCC cells (769P and 7860) and was increased after treatment (Ttest, *P Val<0.05, N=2). Exposure time was 4 hours, mimicking bioavailability curves with IV AA, followed by 24 hour incubation with fresh media, prior to harvesting the cells for nuclear extraction and TET activity analysis. E: 5hmC was measured by LC-ESI-MS/MS and was significantly increased after AA treatment of RCC cells 769P. Addition of catalase did not change the %5hmC (Ttest, * P Val<0.05, N=2). F: Unsupervised clustering based on genome wide methylation analysis conducted by HELP assay. Ward clustering shows global methylation changes are induced by AA treatment. G: Histograms based on methylation (Log (HpaII/MspI)) show increased hypomethylation after AA treatment. H: Smad6 promoter becomes demethylated after AA treatment upon AA treatment in both 786 and 769 ccRCC cells.

FIG. 5A-5E. Fluorescence quenching of recombinant TET-2 protein. A: Fluorescence spectra of 0.5 μM TET-2 are shown after excitation at 280 nm with increasing amounts of Ascorbic acid (from top to bottom). B: The relative fluorescence intensity at 328 nm is shown as a function of Ascorbic acid. A concentration of 132 μM Ascorbic acid reduces the fluorescence signal by 90%. C: Quenching effect on 0.5 μM TET-2 for 2OG (●), L2HG (◯) and for L2HG in presence of 23 μM 2OG (▴). Quenching efficiency of 2OG is higher than L2HG (p<0.001). L2HG quenching is less efficient in presence of 2OG than without (p=0.002). The figure indicates that the substrate specificity of TET-2 is 2OG over L2HG. D: The fluorescence quenching effect of Ascorbic acid on 0.5 μM TET-2 is shown in panel D. Symbols are: (●) TET-2, (□) TET-2 in presence of 100 μM L2HG, (▴) TET-2 in presence of 23 μM 2OG and (□) for the quenching of TET-2 in presence of 23 μM L2HG and 23 μM 2OG. Overlapping of curves (●) and (□) as well as (▴) and (□) taken together, indicates that TET-2 is unaffected by L2HG in the presence of AA. E: comparison of the Stern-Volmer constants +/−SD obtained from the linear range of the data in C & D. P-values are as indicated in the figure.

FIG. 6A-L. AA treatment leads to inhibition of ccRCC growth in vitro and in vivo. A: RCC (786O) cells were treated with AA for 24 hours with and without catalase treatment (to abrogate free radicals). The acute cytotoxicity of AA was reversed by catalase treatment (Ttest, P<0.05, N=2). B,C: RCC Cells were treated with AA and catalase and followed for longer time points. AA led to dose dependent and progressive loss of viability that was not dependent on free radical generation due to catalase treatment (Ttest, P<0.05, N=2). D-F: RCC cells (786O) were treated with Catalase and Ascorbic Acid (AA, 1 mM) and assessed for apoptosis by FACS. AA treatment led to significant increase in apoptosis after 48 hours of treatment. AA exposure for D1 (24 hours) and D3 (48-72 hours) with apoptosis assay at 96 hours. Representative flow figures are shown (TTest, P<0.05, N=2). G-I: RCC cells (786O) were treated with Catalase and Ascorbic Acid (AA, 1 μM) and assessed for cell cycle by FACS. AA treatment led to significant increase in G0/G1 arrest after 48 hours of treatment. AA exposure for D1 (24 hours) and D3 (48-72 hours) with apoptosis assay at 96 hours. Representative flow figures are shown (TTest, P<0.05, N=2). J: RCC cells (786O) were xenografted into immunodeficient NSG mice. After tumors were established, treatment was initiated with IV AA (1 mg/kg/d) or vehicle and tumor measurements were conducted. K, L: AA treatment led to significantly delayed tumor growth (B,C) (TTest, P<0.05, Means+/−S.E.M; N=10 in each cohort).

### DETAILED DESCRIPTION

This document provides methods and materials involved in identifying and/or treating mammals having a cancer (e.g., a ccRCC) based, at least in part on, 5hmC levels within cancer cells. For example, this document provides methods and materials for administering a high dose of AA with or without an additional chemotherapeutic agent or targeted therapy to a mammal identified as having cancer having a reduced level of 5hmC (e.g., “absent,” “low,” “mild,” or “moderate” intensity, correlating with less than 90 percent of cancer cells are positive for 5hmC or 5hmC expression) as well as methods and materials for administering a chemotherapeutic agent without administering a high dose of AA to a mammal identified as having cancer exhibiting 5hmC expression (e.g., greater than 90 percent of cancer cells are positive for 5hmC or 5hmC expression). In some cases, a mammal (e.g., a human) identified as having cancer exhibiting 5hmC expression (e.g., greater than 90 percent of cancer cells are positive for 5hmC or 5hmC expression) can be treated or monitored in a manner that avoids the use of high dose of AA, that avoids the use of any chemotherapeutic agents, and/or that avoids the use of a targeted therapy.

Any appropriate mammal can be identified as having a cancer with a reduced level of 5hmC or as having a cancer without a reduced level of 5hmC. For example, humans and other primates such as monkeys can be identified as having a cancer with a reduced level of 5hmC or as having a cancer without a reduced level of 5hmC. In some cases, dogs, cats, horses, cows, pigs, sheep, mice, or rats can be identified as having a cancer with a reduced level of 5hmC or as having a cancer without a reduced level of 5hmC as described herein.

Any appropriate cancer can be assessed as described herein to determine whether it has a reduced level of 5hmC or does not have a reduced level of 5hmC. For example, kidney cancer (e.g., ccRCC), lymphoma, Myelodysplastic syndromes, Chronic Myelomonocytic leukemia, paraganglioma, pheochromocytoma, can be assessed as described herein to determine whether it has a reduced level of 5hmC or does not have a reduced level of 5hmC.

As described herein, a mammal (e.g., a human) can be identified as having a cancer with a reduced level of 5hmC or without a reduced level of 5hmC by determining the percentage of cancer cells (e.g., from a cancer biopsy) that stain positive for 5hmC using an anti-5hmC antibody. Examples of anti-5hmC antibodies that can be used to determine the percentage of cancer cells positive for 5hmC include, without limitation, MABE1093 (obtained from Millipore Sigma; Catalog No. MABE1093) and 5-Hydroxymethylcytosine (5-hmC) antibody (mAb), clone 59.1 (obtained from Active Motif; Catalog No. 39999).

Once a mammal (e.g., a human) is identified as having a cancer with a reduced level of 5hmC (e.g., less than 90 percent of the cancer cells stain positive for 5hmC using an anti-5hmC antibody), the mammal can be classified as having a cancer having a reduced level of 5hmC. For example, a human identified as having a cancer where less than 90 percent of the cancer cells stain positive for 5hmC using an anti-5hmC antibody can be classified as having cancer with a reduced level of 5hmC. As described herein, a mammal (e.g., a human) identified as having a cancer with a reduced level of 5hmC can be treated with a high dose of AA with or without an additional chemotherapeutic agent or targeted therapy agent. For example, a mammal (e.g., a human) identified as having a cancer with a reduced level of 5hmC as described herein can be administered (a) a high dose of AA as the sole active ingredient administered against the cancer or (b) a high dose of AA in combination with one or more chemotherapeutic agents or targeted therapy agent active against the cancer. In the setting of surgically resected localized cancer with low 5hmC, treatment options can be ascorbic acid and/or a chemotherapeutic agent and/or a targeted therapy agent.

In some cases, a high dose of AA includes administering at least 10 g of AA orally per day or up 1 g of AA/kg/day intravenously up to three times a week. In some cases, a high dose of AA is administer to a mammal (e.g., a human) identified as described herein via an intravenous administration. Examples of chemotherapeutic agents active against cancer that can be used in combination with a high dose of AA as described herein include, without limitation, radiation therapy (Schoenfeld et al., Cancer Cell, 31(4):487-500 (2017)), temozolomide therapy (Schoenfeld et al., Cancer Cell, 31(4):487-500 (2017)), carboplatin and paclitaxel therapy (Ma et al., Sci. Transl. Med., 6(222):222ra18 (2014)), gemcitidine therapy (Monti et al., PLoS ONE, 7(1):e29794 (2012); Polireddy et al., Scientific Reports, 7(1):17188 (2017); and Welsh et al., Cancer Chemother. Pharmacol., 71(3):765-75 (2013)), and erolotinib therapy (Monti et al., PLoS ONE, 7(1):e29794 (2012)). Examples chemotherapeutic agents that can be used to treat cancer as described herein include, without limitation, cisplatin, carboplatin, gemcitabine, etoposide, temozolamide, paclitaxel, 5-FU, and oxaliplatin.

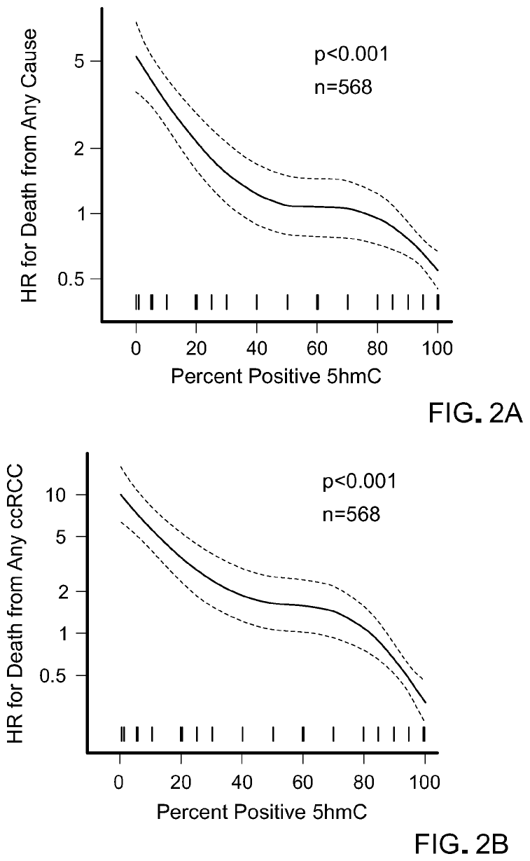

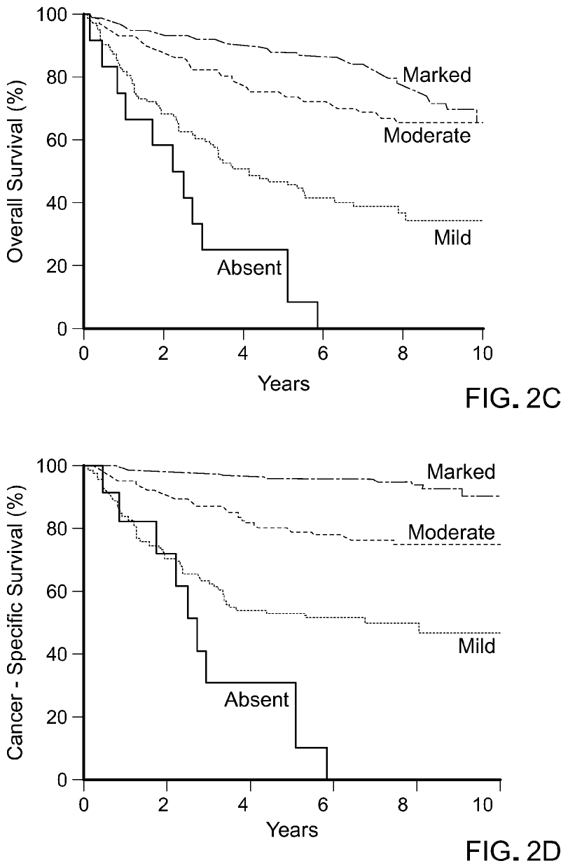

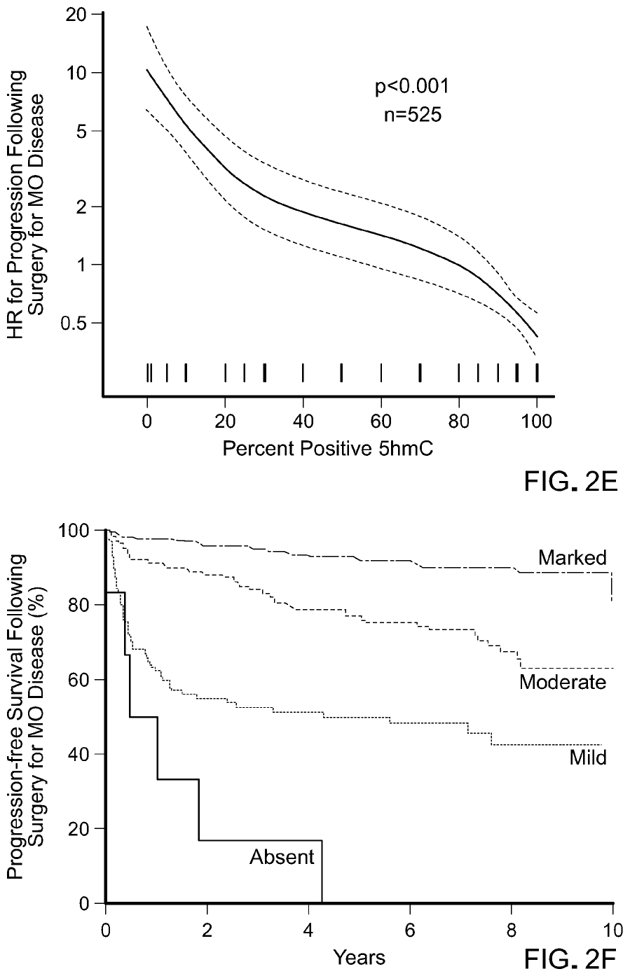

Once a mammal (e.g., a human) is identified as having a cancer without a reduced level of 5hmC (e.g., greater than 90 percent of the cancer cells stain positive for 5hmC using an anti-5hmC antibody), the mammal can be classified as having a cancer without a reduced level of 5hmC. For example, a human identified as having a cancer where greater than 90 percent of the cancer cells stain positive for 5hmC using an anti-5hmC antibody can be classified as having cancer without a reduced level of 5hmC. As described herein, if a mammal (e.g., a human) is identified as having a localized cancer without a reduced level of 5hmC, it allows clinicians and patients to avoid the unnecessary use of a high dose AA in the adjuvant setting (e.g., after surgical resection of localized cancer). In some cases, a mammal identified as having a localized cancer without a reduced level of 5hmC can avoid the unnecessary use a chemotherapeutic agent or a targeted therapy in the treatment of the cancer. The reason is that mammals with cancers having “marked” expression have excellent prognosis after complete resection (see, e.g., FIG. 2F). For example, a mammal (e.g., a human) identified as having a cancer without a reduced level of 5hmC as described herein can be administered one or more chemotherapeutic agents active against the cancer without administering a high dose of AA. Examples of chemotherapeutic agents active against cancer that can be used in the absence of a high dose of AA as described herein include, without limitation, radiation therapy, temozolomide therapy, carboplatin and paclitaxel therapy, gemcitidine therapy, and erolotinib therapy.

One or more chemotherapeutic agents active against a cancer, whether or not used in combination with a high dose of AA, can be administered to a mammal once or multiple times over a period of time ranging from days to months or years. In some cases, one or more chemotherapeutic agents can be formulated into a pharmaceutically acceptable composition for administration to a mammal. For example, a therapeutically effective amount of temozolomide or gemcitidine can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing one or more chemotherapeutic agents active against a cancer can be designed for oral or parenteral (including subcutaneous, intramuscular, intravenous, and intradermal) administration. When being administered orally, a pharmaceutical composition can be in the form of a pill, tablet, or capsule. Compositions suitable for parenteral administration include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

In some cases, a pharmaceutically acceptable composition including one or more chemotherapeutic agents can be administered locally or systemically. For example, a composition provided herein can be administered locally by intravenous injection or blood infusion. In some cases, a composition provided herein can be administered systemically, orally, or by injection to a mammal (e.g., a human).

Effective doses can vary depending on the severity of the cancer, the route of administration, the age and general health condition of the subject, excipient usage, the possibility of co-usage with other therapeutic treatments, and the judgment of the treating physician.

An effective amount of a composition containing one or more chemotherapeutic agents or targeted therapy agents described herein can be any amount that reduces the number of cancer cells within a mammal (e.g., a human) without producing severe toxicity to the mammal. For example, an effective amount of Sunitinib can be from about 25 mg to 50 mg daily (e.g., 4 weeks on, 2 weeks off). If a particular mammal fails to respond to a particular amount, then the amount of the chemotherapeutic agent can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration of a chemotherapeutic agent or a targeted therapy described herein can be any amount that reduces the number of cancer cells within a mammal (e.g., a human) without producing significant toxicity to the mammal. For example, the frequency of administration of temozolomide or gemcitidine can be from about once a day to about once a month (e.g., from about once a week to about once every other week). The frequency of administration of a chemotherapeutic agent described herein can remain constant or can be variable during the duration of treatment. A course of treatment with a composition containing a chemotherapeutic agent described herein can include rest periods. For example, a composition containing one or more chemotherapeutic agents described herein can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cancer) may require an increase or decrease in administration frequency.

An effective duration for administering a composition containing one or more chemotherapeutic agents described herein can be any duration that reduces the number of cancer cells within a mammal (e.g., a human) without producing significant toxicity to the mammal. In some cases, the effective duration can vary from several days to several months. Multiple factors can influence the actual effective duration used for a particular treatment. For example, an effective duration can vary with the frequency of administration, effective amount, use of multiple treatment agents, route of administration, and severity of the condition being treated.

In some cases, a course of treatment and/or the severity of one or more symptoms related to the condition being treated (e.g., cancer) can be monitored. Any appropriate method can be used to determine whether or not a mammal having cancer is being treated. For example, clinical scanning techniques can be used to determine the presence or absence of cancer within a mammal (e.g., a human) being treated.

The invention will be further described in the following examples, which do not limit the scope of the invention described in the claims.

### EXAMPLES

### Example 1—Loss of Hydroxymethylcytosine is an Independent Adverse Prognostic Factor in Clear Cell Renal Cell Carcinoma (ccRCC) and can be Abrogated by Ascorbic Acid Mediated TET Activation

### Cell Lines

RCC cell lines 786-O and 769-P were purchased from the American Type Culture Collection (ATCC). Cell line authentication was done at ATCC. Cells were cultured in RPMI-1640 media supplemented with 10% v/v Fetal Bovine Serum (FBS) and 1% v/v Penicillin/Streptomycin.

Immunohistochemistry (5hmC)

Tissue sectioning and IHC staining was performed at the Pathology Research Core (Mayo Clinic, Rochester, Minn.) using the Leica Bond RX stainer (Leica). Formalin-fixed paraffin-embedded tissues were sectioned at 5 microns, and IHC staining was performed on-line. Slides were retrieved for 20 minutes using Epitope Retrieval 1 (Citrate; Leica) and incubated in protein block (Rodent Block M, Biocare) for 30 minutes. The 5hmc primary antibody (Active Motif) was diluted to 1:1500 in Background Reducing Diluent (Dako) and incubated for 15 minutes.

The detection system used was Polymer Refine Detection System (Leica). Immunostaining visualization was achieved by incubating slides 10 minutes in DAB and DAB buffer (1:19 mixture) from the Bond Polymer Refine Detection System. To this point, slides were rinsed between steps with 1× Bond Wash Buffer (Leica). Slides were counterstained for five minutes using Schmidt hematoxylin and molecular biology grade water (1:1 mixture), followed by several rinses in 1× Bond wash buffer and distilled water; this is not the hematoxylin provided with the Refine kit. Once the immunochemistry process was completed, slides were removed from the stainer and rinsed in tap water for five minutes. Slides were dehydrated in increasing concentrations of ethyl alcohol and cleared in 3 changes of xylene prior to permanent coverslipping in xylene-based medium.

### Patient Selection

The Mayo Clinic Nephrectomy Registry was queried to identify 631 adults treated with radical or partial nephrectomy for sporadic, unilateral, non-cystic ccRCC. Of these, 576 (91%) had 5hmC expression available for analysis.

### Statistical Methods

The clinical and pathologic features studied were summarized with medians and interquartile ranges (IQRs) or frequency counts and percentages and included age at surgery, sex, symptoms at diagnosis, Eastern Cooperative Oncology Group (ECOG) performance status, Charlson score, tumor size, the 2010 primary tumor, regional lymph node, and distant metastases classifications, WHO/ISUP grade, coagulative tumor necrosis, sarcomatoid differentiation, the SSIGN score (Frank et al., J. Urology, 168(6):2395 (2002)) and the progression score (Leibovich et al., Cancer, 97(7):1663 (2003)). Patients with a palpable flank or abdominal mass, discomfort, gross hematuria, acute onset varicocele, or constitutional symptoms including rash, sweats, weight loss, fatigue, early satiety, and anorexia were considered symptomatic. Associations of 5hmC expression with the clinical and pathologic features studied were evaluated using Spearman rank correlation coefficients, Kruskal-Wallis tests, and Wilcoxon rank sum tests. Overall survival, cancer-specific survival, and progression-free survival following surgery were calculated using the Kaplan-Meier method. Progression was defined as distant metastases or death from RCC based on the death certificate in the absence of documented distant metastases. Associations of 5hmC expression with time to death from any cause, time to death from RCC, and time to progression were evaluated using Cox proportional hazards regression models and summarized with hazard ratios (HRs) and 95% confidence intervals (CIs). Statistical analyses were performed using SAS version 9.4 (SAS Institute; Cary, N.C.) and R version 3.2.3 (R Foundation for Statistical Computing; Vienna, Austria). All tests were two-sided and p-values <0.05 were considered statistically significant.

### Measurement of L-2HG by Mass Spectrometry

The enantiomers L-2-hydroxyglutarate and D-2-hydroxyglutarate were measured as described elsewhere (Shim et al., Cancer Discov., 4(11):1290-8 (2014); Jones et al., Methods Mol. Biol., 1633:219-234 (2017); Rakheja et al., Pediatr. Blood Cancer, 56(3):379-83 (2011); Rogers et al., Pediatr. Dev. Pathol., 13(5):408-11 (2010)). Briefly, the cellular extracts were derivatized with (+)-Di-O-acetyl-L-tartaric anhydride (DATAN), a chiral derivatizing agent, followed by liquid chromatography-tandem mass spectrometry (LC-MS/MS). Deuterated stable-isotope, D,L-[3,3,4,4-²H₄]-2-hydroxyglutarate, was used as internal standard, and the results were normalized to protein content of the cell extracts.

### Nuclear Protein Extraction and In-Vitro TET Enzymatic Activity Analysis

Cells were treated with different concentrations of pH neutralized AA, with or without catalase. Exposure time to 1 mM AA was 2 hours (taking into account the plasma concentration curve with intravenous (i.v.) AA and potential differences between plasma concentrations with that of tumor microenvironment) followed by the cells being washed and incubated in fresh media for 24 hours. Nuclear protein was then isolated from cells using the EpiQuik nuclear extraction kit (Epigentek Group Inc, Farmingdale, N.Y., USA), according to the manufacturer's instructions. TET enzymatic activity was measured by using the Enzyme Linked ImmunoSorbent Assay (ELISA)-based Epigenase 5mC Hydroxylase TET Activity/Inhibition Assay Kit (Fluorometric) according to the manufacturer's instructions. This technique relied on the conversion of methylated products at the bottom of the wells to hydroxymethylated products by the TET enzyme present in the nuclear extract. Thus, the amount of hydroxymethylated products formed was a measure of the TET activity of the nuclear extract harvested from the cells being tested. Incubation time of nuclear lysates was 90 minutes. Six micrograms of nuclear lysate was used per well for measurement of TET activity.

### Measurement of 5hmC Levels by Mass Spectrometry

DNA hydrolysis was performed as described elsewhere (Figueroa et al., Cancer Cell, 18(6):553-67 (2010)). Briefly, 1 μg of genomic DNA was first denatured by heating at 100° C. Five units of Nuclease P1 (Sigma-Aldrich, St Louis, Mo., USA, Cat #N8630) were added, and the mixture incubated at 45° C. for 1 hour. A 1/10 volume of 1 M ammonium bicarbonate and 0.002 units of venom phosphodiesterase 1 (Sigma-Aldrich, Cat #P3243) were added to the mixture, and the incubation continued for 2 hours at 37° C. Next, 0.5 units of alkaline phosphatase (Invitrogen, Carlsbad, Calif., USA, Cat #18009-027) were added, and the mixture incubated for 1 hour at 37° C. Quantification was performed using a Liquid Chromatography Electrospray Ionization Tandem Mass Spectrometry (LC-ESI-MS/MS) system in the multiple reaction monitoring mode with some modifications. Before injection into the Zorbax Eclipse Plus C18 2.1 mm×150 mm column (1.8 μm particle size) (Agilent, Santa Clara, Calif., USA, Cat #959759-902), the reactions were diluted 10-fold to dilute out the salts and the enzymes. Samples were analyzed on an Agilent 1290 series liquid chromatography instrument in tandem with the Agilent 6490 triple quadrupole mass spectrometer.

### Genome Wide DNA Methylation Analysis Using the HELP Assay

The HELP assay was carried out as described elsewhere (Sanchari Bhattacharyya et al., Nucleic Acids Res., 41(16):e157 (2013)). Intact DNA of high molecular weight was corroborated by electrophoresis on 1% agarose gel in all cases. One microgram of genomic DNA was digested overnight with either HpaII or MspI (NEB, Ipswich, Mass.). The following day, the reactions were extracted once with phenol-chloroform and resuspended in 11 μL of 10 mM Tris-HCl pH 8.0. The digested DNA was used to set up an overnight ligation of the JHpaII adapter using T4 DNA ligase. The adapter-ligated DNA was used to carry out the PCR amplification of the HpaII and MspI-digested DNA as described elsewhere (Sanchari Bhattacharyya et al., Nucleic Acids Res., 41(16):e157 (2013)). Both amplified fractions were submitted for labeling and hybridization onto a human hg18 custom-designed oligonucleotide array (50-mers) covering 1.3 million HpaII amplifiable fragments (HAF). All microarray hybridizations were subjected to extensive quality control. Uniformity of hybridization was evaluated using a modified version of an algorithm adapted for the NimbleGen platform, and any hybridization with strong regional artifacts was discarded. Bioinformatic analysis was done as described elsewhere (Bhattacharyya et al., Nucleic Acids Res., 41(16):e157 (2013); Suzuki et al., Genome Biol., 11(4):R36 (2010)).

### Cell Viability

Cells were incubated at varying concentrations and at various time periods with L-ascorbic acid (Sigma-Aldrich, Cat #50-81-7) with or without catalase at 100 μg/mL (Sigma-Aldrich, Cat #9001-05-2). Viability was assessed by addition of Cell Titer Blue (Promega, Madison, Wis., USA) and measured via Fluostar Omega Microplate reader (BMG Labtech, Offenburg, Germany). Antioxidant drugs were found to interfere with cell viability measurements by assays that rely on the reducing property of viable cells. They directly reduced the reagent substrate to the reduced fluorescent form, giving spurious results. A protocol modification to counter this interference was done as described elsewhere (Shenoy et al., Laboratory Investigation, 97:494-497 (2017)) and used in this study.

### Flow Cytometry for Apoptosis and Cell Cycle Analysis

786-O cells were treated with high dose ascorbic acid (+catalase), and apoptosis and cell cycle dynamics were studied at the 96 hour time point. Cells were washed with Annexin buffer solution and stained with both propidium iodide (PI) and FITC-Annexin V (Life Technologies), and assayed on a BD FACS Calibur flow cytometer (BD Biosciences). Apoptosis results were analyzed with BD CellQuest software. For cell cycle analysis, cells were fixed with 70% cold ethanol and subsequently analyzed after PI staining. Analysis was performed using FlowJo software.

In Vivo Studies with AA

RCC cells (786O) were xenografted into immunodeficient NSG mice. After tumors were established, treatment was initiated with tail vein injections: IV AA at 1 g/kg/d or vehicle over 5 weeks (5 days dosing/week), and tumor measurements were conducted.

### Fluorescence Spectroscopy

All fluorescence measurements were performed at 20° C. on a Horiba Jobin-Yvon Fluorolog 3 spectrofluorometer equipped with a Wavelength electronics Model LFI-3751 temperature controller. Protein fluorescence emission spectra of TET2 were averaged three times between 305 and 400 nm with excitation at 280 nm. The step width was 1 nm, and the integration time was 1 second.

All protein solutions contained 0.5 μM TET2 in PBS buffer. PBS-buffered stock solutions of 2OG, L2HG, and Ascorbic Acid were added stepwise to the protein solutions prior to the measurement of the fluorescence spectra.

Quenching constants were obtained using the Stern-Volmer equation (Lakowicz and Barry, Principles of Fluorescence Spectroscopy, Third Edition, J. Biomedical Optics, 13(2):029901 (2008)).

F⁰/F=1+K_{D}*[Q]

In this equation, K_{D} was the quenching constant, and [Q] was a defined concentration of ascorbic acid. F⁰ and F were the fluorescence intensities (at 328 nm) in absence and presence of ascorbic acid, respectively.

### Results

Loss of 5hmC is Significantly Associated with Advanced and Higher Grade Clear Cell RCCs

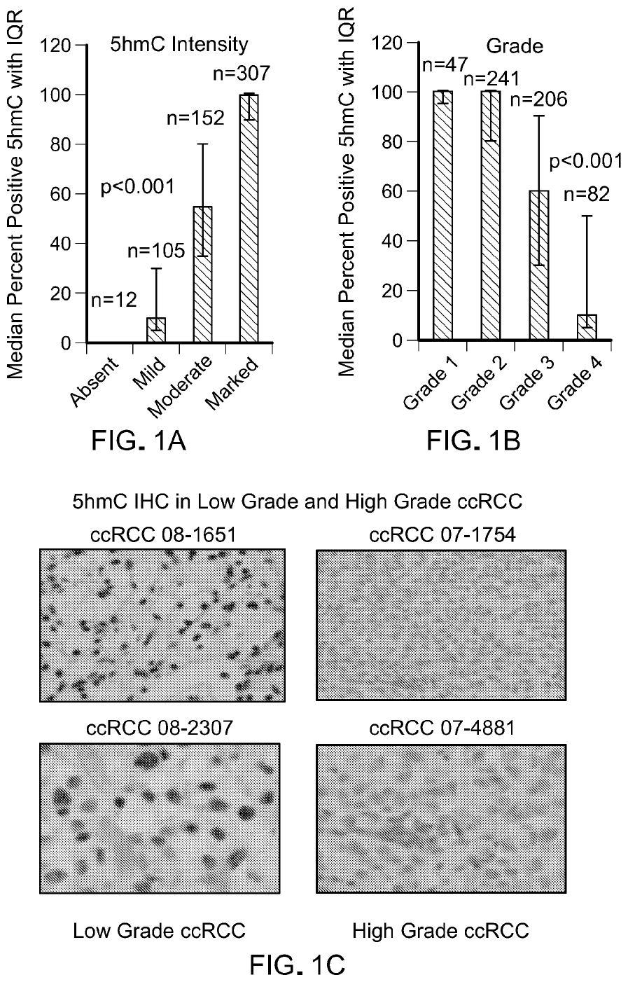

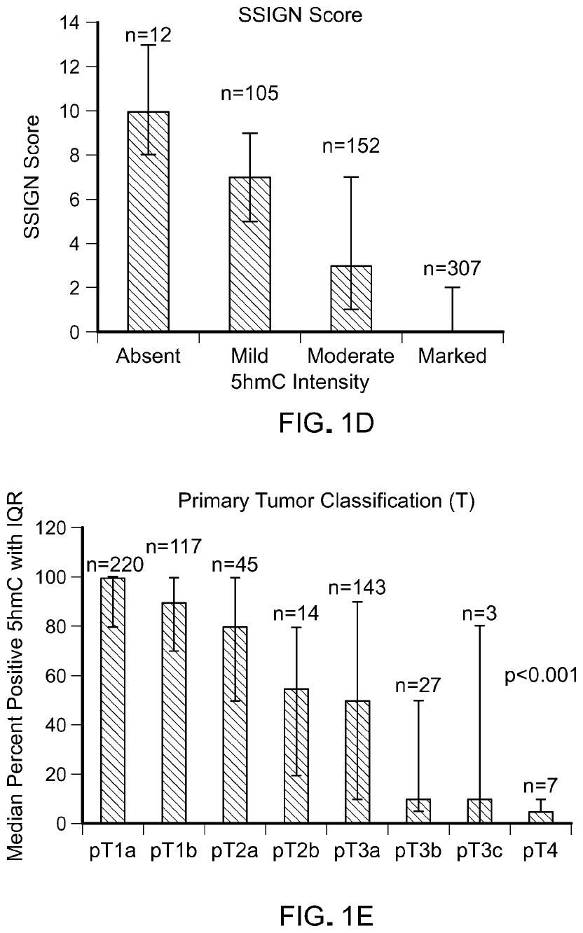

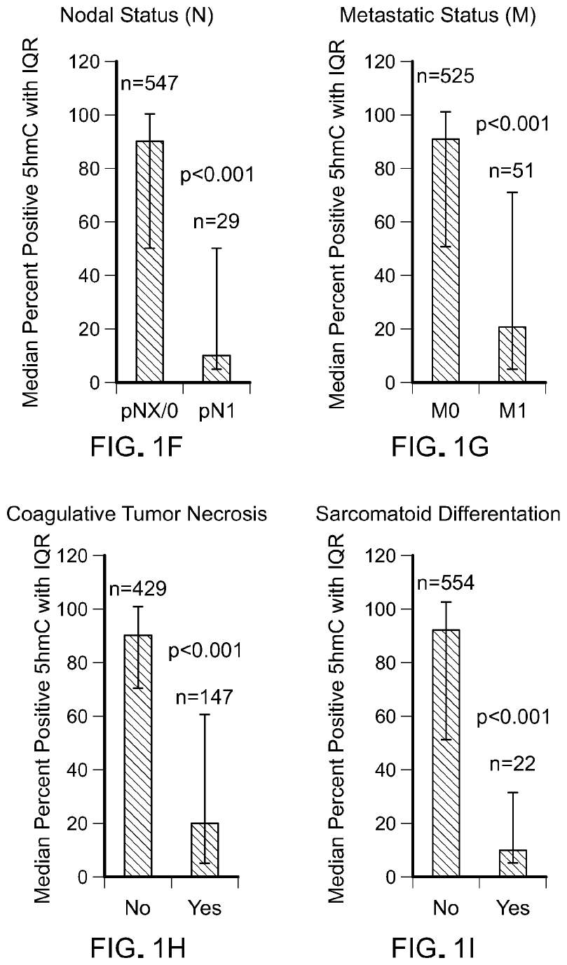

The following was performed to determine whether changes in 5hmC are seen in ccRCC tumors and correlate with any clinicopathologic characteristics. Immunohistochemical evaluation of 5hmC was conducted on a large cohort (n=576) of ccRCC patients. The percent of tumor cells positive for 5hmC correlated well with the intensity of the stain (FIG. 1A).

Pathologically higher grade ccRCC tumors exhibited a striking loss of 5hmC compared with lower grade tumors (FIGS. 1B and 1C). Median percent positive 5hmC for grades 1, 2, 3, and 4 tumors were 100%, 100%, 60%, and 10%, respectively (p<0.001) (FIG. 1B). Loss of 5-hmC also was associated with a higher primary tumor classification, nodal and systemic metastasis (FIGS. 1D-1F, p<0.001). Tumor size negatively correlated with percent positive 5hmC (correlation coefficient=−0.52, p<0.001), and median sizes for tumors with absent, mild, moderate, and marked 5hmC intensity were 11.1, 9.4, 6.2, and 3.6 cm, respectively (p<0.001). The percentages of absent, mild, moderate, and marked 5hmC intensity tumors that were grade 4 were 50%, 45%, 12%, and 4%, respectively (p<0.001). Tumors with additional signs of aggressiveness such as coagulative tumor necrosis and sarcomatoid differentiation also were associated with significantly lower percent positive 5hmC (FIGS. 1G-1H). Tables 1-3 show associations of percent positive 5hmC and 5hmC intensity with clinical and pathologic features. Taken together, these data indicated that a loss of 5hmC is associated with a clinicopathological advanced phenotype of ccRCC. The prognostic value of loss of 5hmC was investigated in a univariable and multivariable setting.

TABLE 1
Summary of clinical and pathologic features and 5hmC expression

| Feature | Median (IQR) | |
|---|---|---|
| Age at surgery in years | 61 | (53-69) |
| Charlson score | 1 | (0-2) |
| Tumor size in cm | 5.0 | (3.0-8.5) |
| SSIGN scare | 2 | (0-6) |
| Percent positive 5hmC | 90 | (40-100) |
| | N (%) | |
| Sex | | |
| Female | 216 | (38) |
| Male | 360 | (62) |
| Symptoms | 254 | (44) |
| Constitutional symptoms | 100 | (17) |
| ECOG performance states | | |
| 0 | 479 | (83) |
| 1 | 59 | (10) |
| 2 | 27 | (5) |
| 3 | 11 | (2) |
| 2010 pT | | |
| pT1a | 220 | (38) |
| pT1b | 117 | (20) |
| pT2a | 45 | (8) |
| pT2b | 14 | (2) |
| pT3a | 143 | (25) |
| pT3b | 27 | (5) |
| pT3c | 3 | (1) |
| pT4 | 7 | (1) |
| 2010 pN | | |
| pNX/0 | 547 | (95) |
| pN1 | 29 | (5) |
| 2010 M | | |
| M0 | 525 | (91) |
| M1 | 51 | (9) |
| Grade | | |
| 1 | 47 | (8) |
| 2 | 241 | (42) |
| 3 | 206 | (36) |
| 4 | 82 | (14) |
| Coagulative tumor necrosis | 147 | (26) |
| Sarcomatoid differentiation | 22 | (4) |
| 5hmC intensity | | |
| Absent | 12 | (2) |
| Mild | 105 | (18) |
| Moderate | 152 | (26) |
| Marked | 307 | (53) |

TABLE 2
Associations of percent positive 5hmC
with clinical and pathologic features

| Feature | Correlation* | P-value |
|---|---|---|
| Age at surgery in years | −0.07 | 0.092 |
| Charlson score | −0.12 | 0.004 |
| Tumor size in cm | −0.52 | <0.001 |
| SSIGN score | −0.61 | <0.001 |
| | Median (IQR)^{†} | |

| | | | |
|---|---|---|---|
| Sex | | | |
| Female | 90 | (50-100) | 0.016 |
| Male | 80 | (30-100) | |
| Symptoms | | | |
| No | 90 | (70-100) | <0.001 |
| Yes | 60 | (10-90) | |
| Constitutional symptoms | | | |
| No | 90 | (50-100) | <0.001 |
| Yes | 30 | (5-80) | |
| ECQG performance status | | | |
| 0 | 90 | (50-100) | 0.006 |
| ≥1 | 70 | (10-100) | |
| 2010 pT | | | |
| pT1a | 100 | (80-100) | <0.001 |
| pT1b | 90 | (70-100) | |
| pT2a | 80 | (50-100) | |
| pT2b | 55 | (20-80) | |
| pT3a | 50 | (10-90) | |
| pT3b | 10 | (5-50) | |
| pT3c | 10 | (0-80) | |
| pT4 | 5 | (5-10) | |
| 2010 pN | | | |
| pNX/0 | 90 | (50-100) | <0.001 |
| pN1 | 10 | (5-50) | |
| 2010 M | | | |
| M0 | 90 | (50-100) | <0.001 |
| M1 | 20 | (5-70) | |
| Grade | | | |
| 1 | 100 | (95-100) | <0.001 |
| 2 | 100 | (80-100) | |
| 3 | 60 | (30-90) | |
| 4 | 10 | (5-50) | |
| Coagulative tumor necrosis | | | |
| No | 90 | (70-100) | <0.001 |
| Yes | 20 | (5-60) | |
| Sarcomatoid differentiation | | | |
| No | 90 | (50-100) | <0.001 |
| Yes | 10 | (5-30) | |
| 5hmC intensity | | | |
| Absent | 0 | (0-0) | <0.001 |
| Mild | 10 | (5-30) | |
| Moderate | 55 | (35-80) | |
| Marked | 100 | (90-100) | |

*Spearman rank correlation coefficient.
^{†}Median (IQR) percent passive 5hmC.

TABLE 3
Associations of 5hmC intensity with clinical and pathologic features

| | 5hmC Intensity | | | | | | | | |
|---|---|---|---|---|---|---|---|---|---|
| | Absent | | Mild | | Moderate | | Marked | | |
| | N = 12 | | N = 105 | | N = 152 | | N = 307 | | |
| Feature | Median (IQR) | | | | | | | | P-value |
| Age at surgery in years | 58 | (51-71) | 62 | (54-70) | 64 | (55-70) | 60 | (51-68) | 0.004 |
| Charlson score | 5 | (0-6) | 1 | (0-3) | 1 | (0-2) | 1 | (0-2) | 0.005 |
| Tumor size in cm | 11.1 | (8.5-16.8) | 9.4 | (6.5-12.0) | 6.2 | (4.0-8.9) | 3.6 | (2.3-5.5) | <0.001 |
| SSIGN score | 10 | (8-13) | 7 | (5-9) | 3 | (1-7) | 0 | (0-2) | <0.001 |
| Percent positive 5hmC | 0 | (0-0) | 10 | (5-30) | 55 | (35-80) | 100 | (90-100) | <0.001 |

| | N (%) | | | | | | | | |
|---|---|---|---|---|---|---|---|---|---|
| Sex | | | | | | | | | |
| Female | 3 | (25) | 34 | (32) | 50 | (33) | 129 | (42) | 0.018 |
| Male | 9 | (75) | 71 | (66) | 102 | (67) | 178 | (58) | |
| Symptoms | 12 | (100) | 75 | (71) | 74 | (49) | 93 | (30) | <0.001 |
| Constitutional symptoms | 5 | (42) | 40 | (38) | 28 | (18) | 27 | (9) | <0.001 |
| ECOG performance status | | | | | | | | | |
| 0 | 11 | (92) | 79 | (75) | 126 | (83) | 263 | (86) | 0.051 |
| ≥1 | 1 | (8) | 26 | (25) | 26 | (17) | 44 | (14) | |
| 2010 pT | | | | | | | | | |

| | | | | | | | | |
|---|---|---|---|---|---|---|---|---|
| pT1a | 0 | 13 | (12) | 38 | (25) | 169 | (55) | <0.001 |

| | | | | | | | | |
|---|---|---|---|---|---|---|---|---|
| pT1b | 1 | (8) | 7 | (7) | 37 | (24) | 72 | (23) |

| | | | | | | | |
|---|---|---|---|---|---|---|---|
| pT2a | 0 | 7 | (7) | 16 | (11) | 22 | (7) |

| | | | | | | | | |
|---|---|---|---|---|---|---|---|---|
| pT2b | 2 | (17) | 3 | (3) | 4 | (3) | 5 | (2) |
| pT3a | 7 | (58) | 48 | (46) | 51 | (34) | 37 | (12) |
| pT3b | 1 | (8) | 20 | (19) | 5 | (3) | 1 | (<1) |

| | | | | | | | |
|---|---|---|---|---|---|---|---|
| pT3c | 1 | (8) | 1 | (1) | 1 | (1) | 0 |

| | | | | | | |
|---|---|---|---|---|---|---|
| pT4 | 0 | 6 | (6) | 0 | 1 | (<1) |

| | | | | | | | | | |
|---|---|---|---|---|---|---|---|---|---|
| 2010 pN | | | | | | | | | |
| pNX/0 | 9 | (75) | 89 | (85) | 147 | (97) | 302 | (98) | <0.001 |
| pN1 | 3 | (25) | 16 | (15) | 5 | (3) | 5 | (2) | |
| 2010 M | | | | | | | | | |
| M0 | 6 | (50) | 66 | (82) | 134 | (66) | 299 | (97) | <0.001 |
| M1 | 6 | (50) | 19 | (18) | 18 | (12) | 8 | (3) | |
| Grade | | | | | | | | | |

| | | | | | | | |
|---|---|---|---|---|---|---|---|
| 1 | 0 | 0 | 1 | (1) | 46 | (15) | <0.001 |

| | | | | | | | | | |
|---|---|---|---|---|---|---|---|---|---|
| 2 | 1 | (8) | 5 | (5) | 44 | (29) | 191 | (62) | |
| 3 | 5 | (42) | 53 | (50) | 89 | (59) | 59 | (19) | |
| 4 | 6 | (50) | 47 | (45) | 18 | (12) | 11 | (4) | |
| Coagulative tumor necrosis | 10 | (83) | 58 | (65) | 50 | (33) | 19 | (6) | <0.001 |
| Sarcomatoid differentiation | 1 | (8) | 14 | (13) | 3 | (2) | 4 | (1) | <0.001 |

Loss of 5hmC is an Independent Adverse Prognostic Factor in Clear Cell RCC and Predicts Shortened Time to Metastatic Disease after Surgical Resection for Localized (M0) Disease

In this cohort of ccRCC cases, 185 patients out of total 576 died at a median of 2.7 years following surgery (IQR 1.1-5.1). The median duration of follow-up for the 391 patients who were still alive at last follow-up was 7.2 years (IQR 6.2-8.7). Eight patients who died from unknown causes were excluded from the analyses of cancer-specific survival. Of the remaining 568 patients, 112 died from RCC at a median of 2.1 years following surgery (IQR 0.9-3.5).

Loss of 5hmC was found to be significantly associated with reduced cancer specific survival in both univariable and multivariable analysis. Associations of 5hmC expression with time to death from any cause and time to death from RCC were summarized in Table 4. The percent positive 5hmC was inversely related to death from any cause (univariable HR for a 10% increase 0.82, 95% CI 0.79-0.85, p<0.001, FIG. 2A) and death from RCC (univariable HR for a 10% increase 0.74, 95% CI: 0.70-0.78, p<0.001, FIG. 2B; multivariable HR 0.93, 95% CI: 0.87-0.98, p=0.013). Patients with absent, mild, and moderate 5hmC tumor staining intensity had a univariable HR of death from any cause of 11.60 (p<0.001), 4.44 (p<0.001), and 1.69 (p=0.007), respectively, compared with marked intensity. The median overall survival (OS) in the absent, mild, and moderate 5hmC intensity cohorts occurred at 2.4, 4.1, and 10.5 years, respectively. Median OS in the marked group has not been reached (FIG. 2C). Patients with absent, mild, and moderate 5hmC tumor staining intensity had a univariable HR of death from RCC of 27.27 (p<0.001), 11.15 (p<0.001), and 4.06 (p<0.001), respectively, compared with marked intensity. The median cancer specific survival (CSS) in the absent and mild intensity cohorts occurred at 2.7 and 6.8 years, respectively. Median CSS in the moderate and marked 5hmC intensity group has not been reached. 10-year CSS in the marked 5hmC intensity group was 90% (FIG. 2D).

TABLE 4
Associations of 5hmC expression with patient outcomes

| | Univariable | | | Multivariable* | | |
|---|---|---|---|---|---|---|
| Feature | HR (95% CI) | | P-value | HR (95% CI) | | P-value |
| | Death from Any Cause | | | | | |
| Percent positive 5hmC^{†} | 0.82 | (0.79-0.85) | <0.001 | 0.97 | (0.93-1.02) | 0.22 |
| 5hmC intensity | | | | | | |
| Absent | 11.60 | (6.19-21.76) | <0.001 | 1.49 | (0.73-3.06) | 0.27 |
| Mild | 4.44 | (3.12-6.31) | <0.001 | 0.96 | (0.62-1.46) | 0.83 |
| Moderate | 1.69 | (1.15-2.46) | 0.007 | 0.73 | (0.49-1.10) | 0.13 |
| Marked | 1.0 | (reference) | | 1.0 | (reference) | |
| | Death from RCC | | | | | |
| Percent positive 5hmC^{†} | 0.74 | (0.70-0.78) | <0.001 | 0.93 | (0.87-0.98) | 0.013 |
| 5hmC intensity | | | | | | |
| Absent | 27.27 | (12.49-59.52) | <0.001 | 1.49 | (0.61-3.63) | 0.38 |
| Mild | 11.15 | (6.50-19.13) | <0.001 | 1.52 | (0.83-2.80) | 0.18 |
| Moderate | 4.06 | (2.29-7.19) | <0.001 | 1.25 | (0.68-2.27) | 0.48 |
| Marked | 1.0 | (reference) | | 1.0 | (reference) | |
| | Progression among M0 Patients | | | | | |
| Percent positive 5hmC^{†} | 0.76 | (0.72-0.80) | <0.001 | 0.91 | (0.86-0.97) | 0.002 |
| 5hmC intensity | | | | | | |
| Absent | 27.07 | (11.06-66.24) | <0.001 | 4.69 | (1.84-11.96) | 0.001 |
| Mild | 8.44 | (5.25-13.56) | <0.001 | 1.43 | (0.80-2.55) | 0.23 |
| Moderate | 3.23 | (1.98-5.27) | <0.001 | 1.23 | (0.72-2.08) | 0.45 |
| Marked | 1.0 | (reference) | | 1.0 | (reference) | |

*Adjusted for age, sex, and SSIGN score for time to death from any cause and time to death from RCC. Adjusted for age, sex, and progression score for time to progression among M0 patients.
^{†}HR and CI represent a 10% increase.

Loss of 5hmC also was associated with progression following surgery for non-metastatic (M0) disease in a large cohort of cases (n=525). Associations of 5hmC with time to progression among M0 patients were summarized in Table 4. There were 6 (1%), 86 (16%), 134 (26%), and 299 (57%) tumors with absent, mild, moderate, and marked 5hmC intensity, respectively. Five patients who died from unknown causes without experiencing distant metastases were excluded from the analyses of progression-free survival. Of the remaining 520 patients, 117 experienced progression at a median of 1.3 years following surgery (IQR 0.3-3.6). The percent positive 5hmC was found to be inversely related to progression following surgery for M0 disease (univariable HR for a 10% increase 0.76, 95% CI: 0.72-0.80, p<0.001, FIG. 2E; multivariable HR 0.91, 95% CI: 0.86-0.97, p=0.002). Patients with absent, mild, and moderate 5hmC tumor staining intensity had a univariable HR of progression following surgery for M0 patients of 27.07 (p<0.001), 8.44 (p<0.001), and 3.23 (p<0.001), respectively, compared with marked intensity. The median PFS in the absent and mild intensity cohorts occurred at 0.8 and 4.3 years, respectively. Median PFS in the moderate and marked 5hmC intensity groups has not been reached. 10-year PFS in the marked 5hmC intensity group was 81% (FIG. 2F). The relationship of absent 5hmC and decreased PFS also was validated in multivariable analysis, with a HR for progression of 4.69 (95% CI: 1.84-11.96, p=0.001).

L-2-Hydroxyglutarate Dehydrogenase (L2HGDH) Deletions and Under-Expression were Seen in ccRCC and Significantly Associated with Hypermethylation and Adverse Prognosis

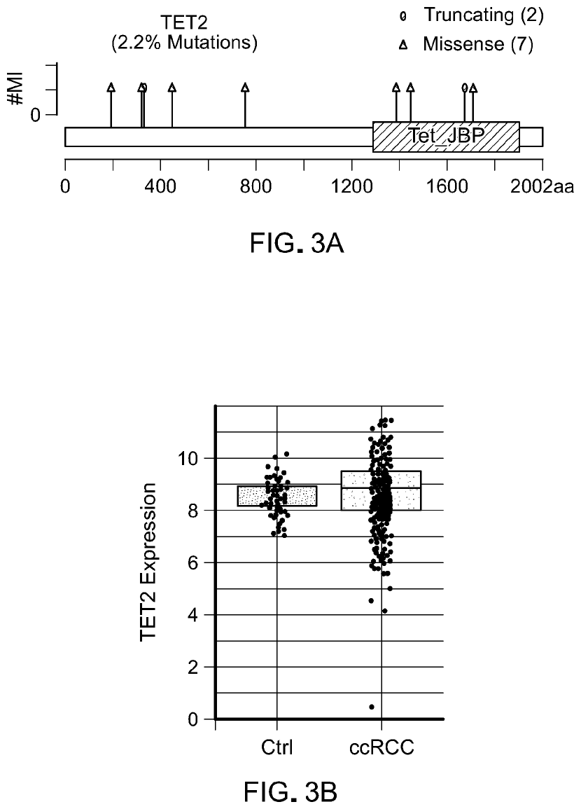

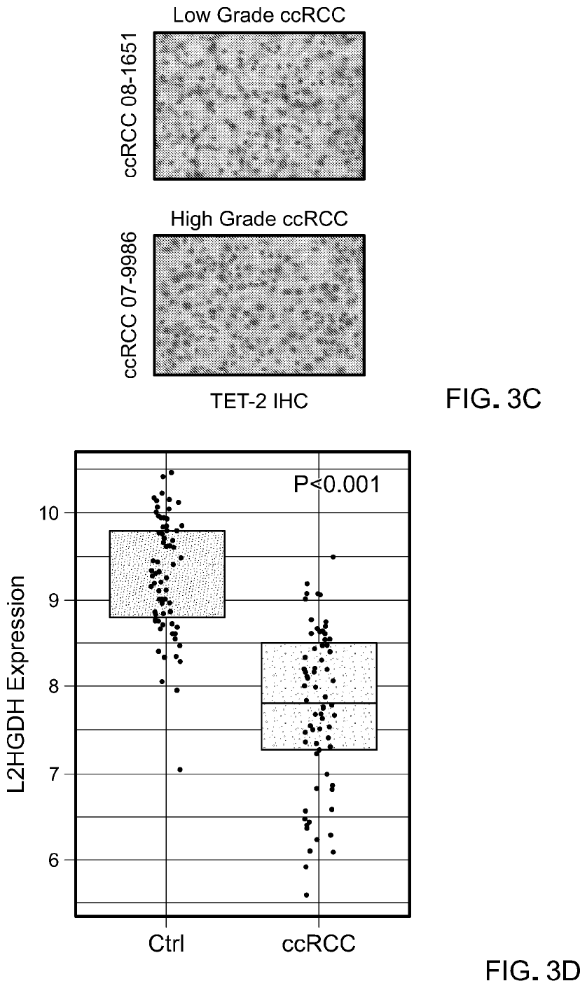

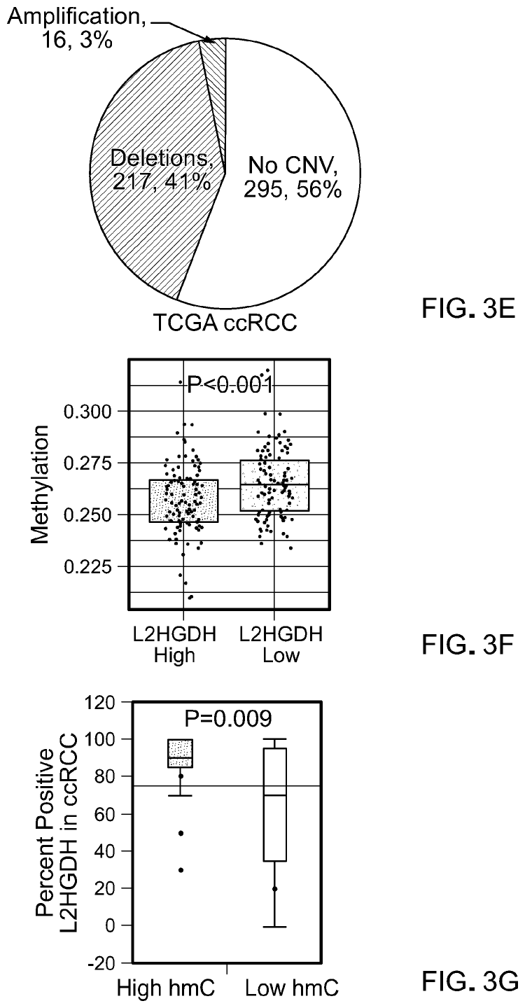

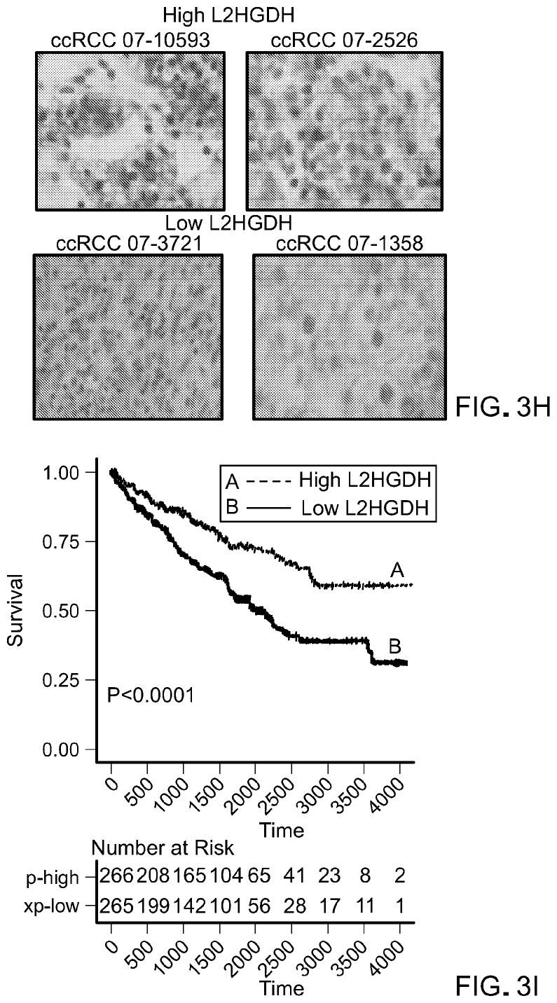

The following was performed to determine the reason for the loss of 5hmC in high grade ccRCC. TET-2 mutations are common in hematologic malignances and are associated with hypermethylation. Analysis of TCGA cohort (n=418) revealed that TET-2 was mutated (heterozygous) only in 2.2% of ccRCC tumors (FIG. 3A). No decrease in TET-2 expression was seen between ccRCC samples and matched controls obtained from TCGA (FIG. 3B). TET-2 immunohistochemistry revealed no difference in expression patterns between high grade and low grade ccRCC (representative pictures in FIG. 3C). Although TET-2 expression was intact, its activity was previously shown to be inhibited by the accumulation of oncometabolite L-2-Hydroxyglutarate (L2HG) in ccRCC (Shim et al., Cancer Discov., 4(11):1290-8 (2014)). Reduced expression of the enzyme, L2HGDH, was reported to be partly responsible for the accumulation of L2HG in ccRCC (Shim et al., Cancer Discov., 4(11):1290-8 (2014)). Consistent with that observation, L2HGDH expression was significantly lower in ccRCC compared to matched normal kidney tissue (TCGA data, P<0.001)(FIG. 3D). Copy number data from TCGA revealed that deletions at the L2HGDH locus were seen in 41% of samples (FIG. 3E). Integration of methylation data with L2HGDH expression determined that ccRCC tumors with lower L2HGDH were significantly associated with higher cytosine methylation (FIG. 3F, P<0.001). L2HGDH IHC from 20 high-5hmC ccRCC and 20-low 5hmC ccRCC samples from this cohort suggested that lower 5hmC levels in ccRCC were associated with lower L2HGDH levels (FIG. 3G, p=0.009), representative pictures illustrated in FIG. 3H. Furthermore, lower L2HGDH expression was associated with worse survival in the cohort of 533 ccRCC patients in the TCGA (FIG. 3I; P-value <0.0001).

AA Treatment Leads to Increased TET Activity, Loss of Methylation and Gain of Hydroxymethyl Cytosine Levels in ccRCC Cells

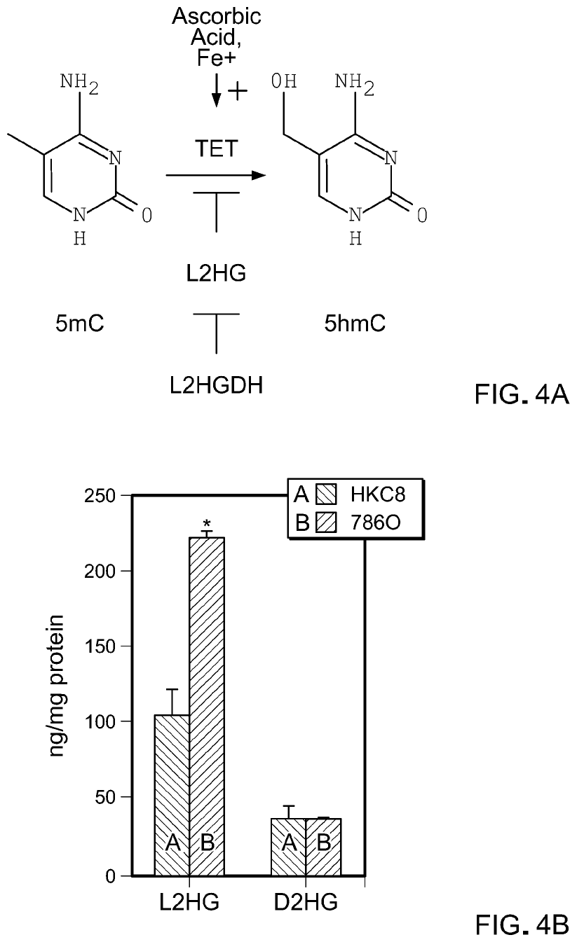

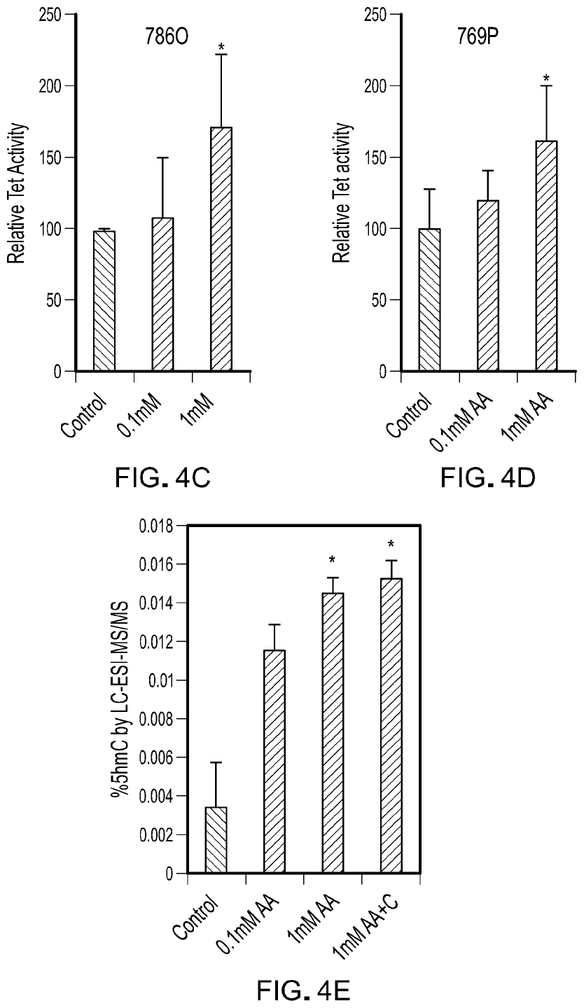

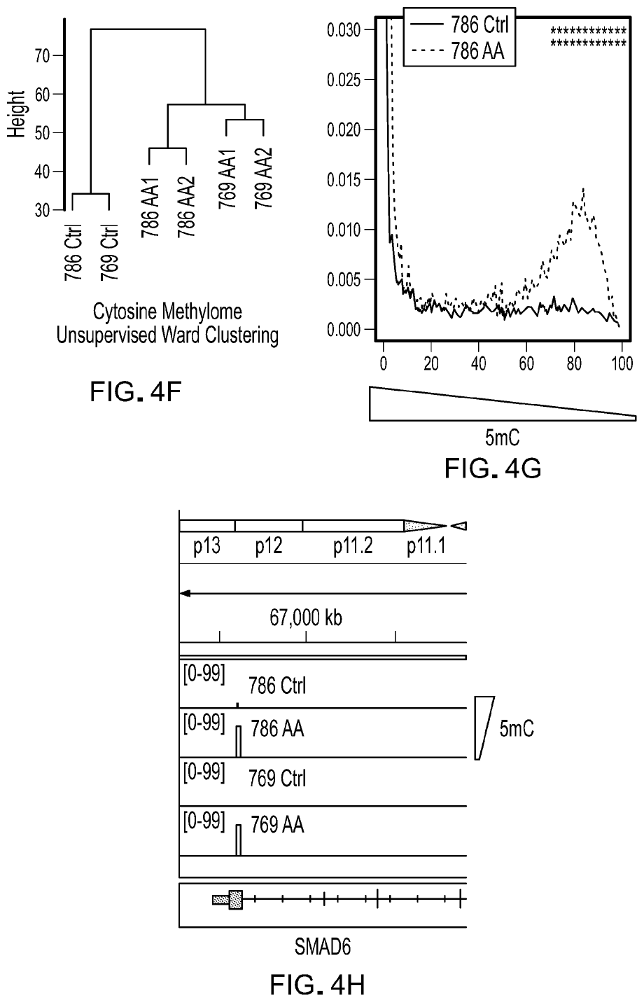

Since lower L2HGDH and consequently higher expression of L2HG can functionally inhibit TET enzymes, pharmacologic activation of TET in ccRCC was evaluated. Ascorbic acid is an essential cofactor for TET enzymes (binds to the catalytic domain and aids in conversion of Fe3+ to Fe2+ that is used by TET enzymes for conversion of methyl to hydroxymethyl cytosines (FIG. 4A) (Cimmino et al., Cell, 170(6):1079-1095 (2017); and Agathocleous et al., Nature, 549(7673):476-481 (2017)). The effects of AA on two ccRCC cell lines that had heterozygous deletions affecting the L2HGDH locus (TCGA data) were evaluated. L2HG levels were raised in 7860 cells when compared to kidney tubular control (FIG. 4B). Exposure of ccRCC cell lines to pH neutralized AA led to an increase in TET enzymatic activity (FIGS. 4C-4D). To determine the consequence of this AA-induced increased TET activity on DNA hydroxymethylation, mass spectrometry (LC-ESI-MS/MS) was performed and revealed an increase in 5hmC after AA treatment (FIG. 4E). Global changes in methylation were validated by the HELP assay that relies on differential restriction digestion of methylated CpGs followed by high throughput sequencing analysis (Bhattacharyya et al., Nucleic Acids Res., 41(16):e157 (2013)). Unsupervised clustering demonstrated that AA treatment led to changes in cytosine methylation patterns with epigenetic dissimilarity between control and AA treated ccRCC cells (FIG. 4F). Qualitatively, AA treatment led to loss of methylation (FIG. 4G) and affected loci that had been previously shown to be hypermethylated in RCC (Smad6 promoter is demethylated after AA treatment, FIG. 4H).

### Fluorescence Quenching of Recombinant TET-2 is Unaffected by L2HG in the Presence of Ascorbic Acid

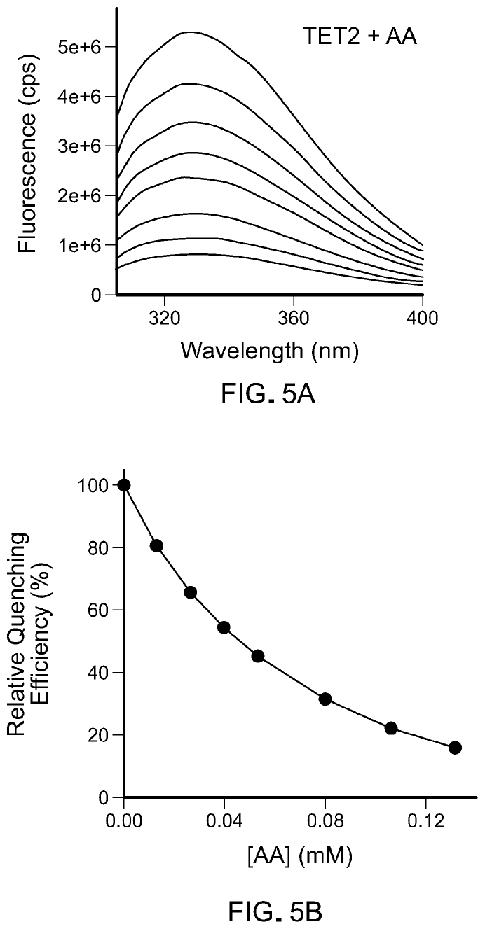

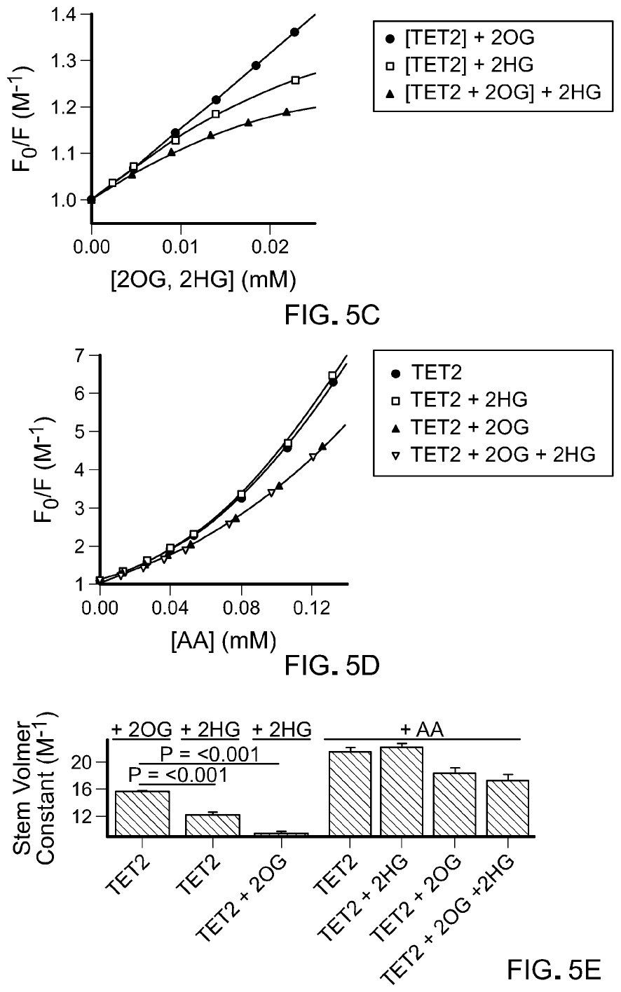

The interaction between the recombinant TET-2 protein with AA and its cofactor 2OG as well as its competitive inhibitor L2HG was evaluated. There was significant quenching of fluorescence when AA was added to a solution containing 0.5 μM TET-2 (FIG. 5A). Maximal quenching was obtained with a dose of 132 μM where a 90% reduction of the fluorescence emission signal was observed (FIG. 5B). This AA concentration would be very difficult to achieve even in the plasma with the maximum tolerated oral dose. However, the relation between intracellular AA concentration in vivo in kidney cancer cells and the plasma AA concentration is unknown. The interaction of TET-2 with 2OG and L2HG was studied (FIGS. 5C and 5E). The quenching efficiency of 2OG (15.66+/−0.12 M⁻¹) was found to be higher than for L2HG (12.15+/−0.51 M⁻¹) yielding a p-value of <0.001. Furthermore, 2OG seemed to prevent L2HG from interacting efficiently, as L2HG quenching was less efficient (9.36+/−0.40 M⁻¹) in presence of 23 μM 2OG than without (p<0.001). Taken together, this indicated that the substrate specificity of TET-2 for 2OG was higher than L2HG.

The quenching of TET-2 with AA was performed, in the presence and absence of L2HG and 2OG (FIGS. 5D and 5E). Addition of AA led to abrogation of decreased quenching that was seen in the presence of L2HG. There was an overlap of the quenching curves of TET-2+AA (21.46+/−0.70 M⁻¹) and TET-2+L2HG+AA (22.17+/−0.68 M⁻¹) as well as an overlap of quenching curves of TET-2+2OG+AA (18.37+/−0.80 M⁻¹) and TET-2+2OG+L2HG+AA (17.34+/−0.87 M⁻¹), suggesting that TET-2 was unaffected by L2HG in the presence of AA.

High Dose AA Treatment Inhibits Growth of ccRCC Cells Via Non Free Radical Mechanisms

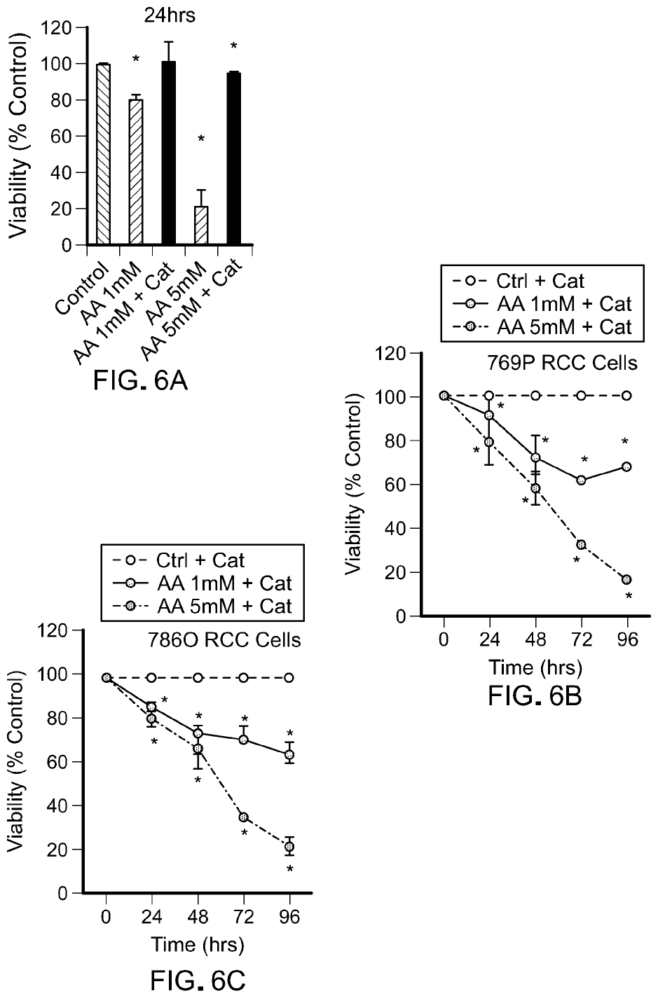

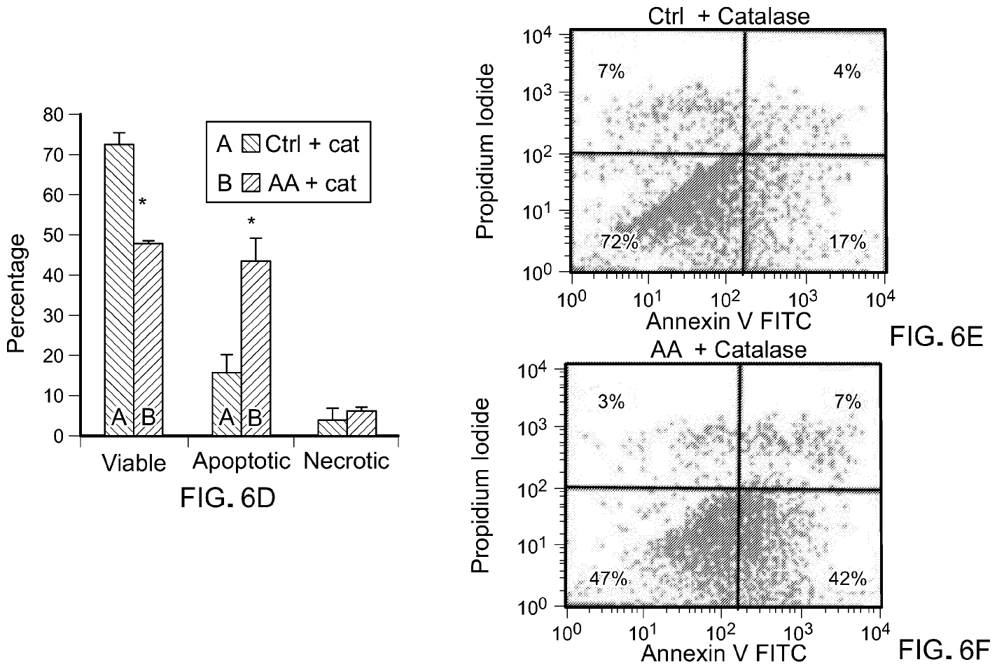

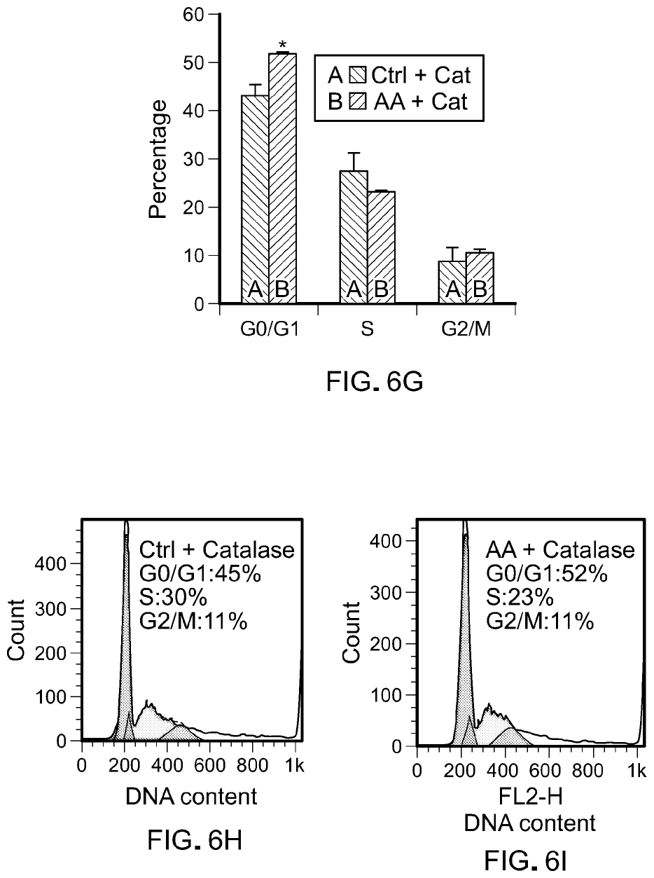

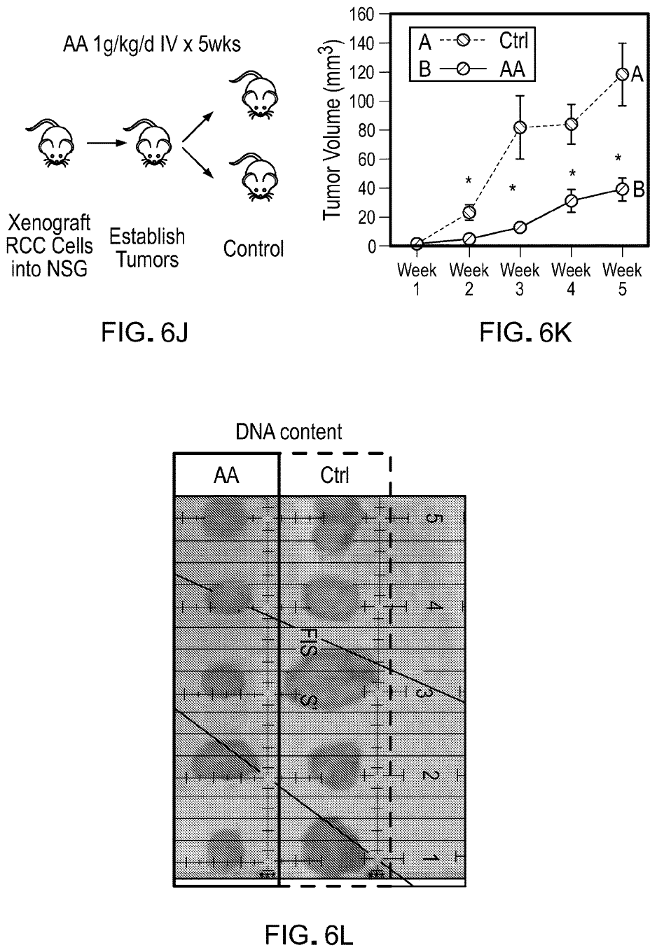

The following was performed to determine if treatment with ascorbic acid could lead to inhibitory effects in ccRCC. AA, when added to culture media, generates hydrogen peroxide (H₂O₂) that can cause cytotoxicity (Chen et al., Proc. Natl. Acad. Sci. USA, 102(38):13604-9 (2005); Chen et al., Proc. Natl. Acad. Sci. USA, 104(21):8749-54 (2007); and Chen et al., Proc. Natl. Acad. Sci. USA, 105(32):11105-9 (2008)). To evaluate the functional impact of epigenetic effects of AA in vitro, ccRCC derived cell lines were treated with short term exposure to high dose ascorbic acid (mimicking the bioavailability curves of IV AA doses currently being used in early phase trials and roughly accounting for difference in plasma concentrations and that of tumor microenvironment) with or without catalase treatment, to counter the H₂O₂ generated by AA. Acute loss in viability was observed with high dose AA that was reversed in the presence of catalase co-treatment, suggesting that the acute cytotoxicity with short term exposure of high dose AA (millimolar concentration) was primarily mediated by H₂O₂, as demonstrated previously (Chen et al., Proc. Natl. Acad. Sci. USA, 102(38):13604-9 (2005); and Chen et al., Proc. Natl. Acad. Sci. USA, 104(21):8749-54 (2007)) (FIG. 6A). However, treatment of high dose AA with catalase resulted in reduced viability that was detected after longer time points in RCC cells, demonstrating that AA could exert anti-tumor effects through non-H₂O₂ mechanisms with a longer-term exposure (FIGS. 6B and 6C).

To determine the mechanism of loss of viability, ccRCC cells were treated with AA and catalase and assessed for apoptosis and cell cycle dynamics. High dose AA treatment at the 96 hour time point led to increased apoptosis in ccRCC cells (FIGS. 6D-F). ccRCC cells also were found to be significantly arrested in G0/G1 stage of cell cycle at 96 hours after high dose AA treatment (FIGS. 6G-I). Next, xenografts from RCC cells were established in immunodeficient NSG mice. The mice were treated for 5 weeks with 1 g/kg of AA given per day for 5 days/week given intravenously or with vehicle control (n=10 in each cohort) (FIG. 6J). Tumor measurements revealed a significantly reduced rate of ccRCC growth with AA treatment when compared to vehicle controls (FIGS. 6K-6L).

These results demonstrate that loss of 5hmC is an independent adverse prognostic factor in ccRCC and that a grading of loss of 5hmC based on intensity into absent, mild, moderate, and marked (with the IHC method described herein) can be used as a strong tool to predict outcomes and can be integrated into prognostic models, therapeutic decisions, and/or clinical trial designs. In addition, these results demonstrate that in RCC the AA treatment leads to increased TET activity, loss of methylation and gain of hydroxymethyl cytosine levels in ccRCC cells and has single-agent anti-tumor activity.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-8. (canceled)

9. A method for treating cancer, wherein said method comprises:
(a) identifying a mammal as having a cancer comprising a reduced level of 5hmC, and
(b) administering a high dose of AA, a chemotherapeutic agent, or a targeted therapy to said mammal.

10. The method of claim 9, wherein said mammal is a human.

11. The method of claim 9, wherein said cancer is a kidney cancer.

12-14. (canceled)

15. The method of claim 9, wherein said method comprises administering said high dose of AA to said mammal.

16. The method of claim 15, wherein said high dose of AA is administered as the sole active ingredient against said cancer.

17. The method of claim 9, wherein said method comprises administering said chemotherapeutic agent to said mammal.

18. The method of claim 17, wherein said chemotherapeutic agent is cisplatin, carboplatin, gemcitabine, etoposide, temozolamide, paclitaxel, 5-FU, or oxaliplatin.

19. The method of claim 17, wherein said chemotherapeutic agent is administered as the sole active ingredient against said cancer.

20. The method of claim 9, wherein said high dose of AA and said chemotherapeutic agent is administered to said mammal.

21. The method of claim 20, wherein said chemotherapeutic agent and said high dose of AA is administered during the same day.

22. The method of claim 20, wherein said chemotherapeutic agent is administered before said high dose of AA.

23. The method of claim 20, wherein said chemotherapeutic agent is administered after said high dose of AA.

24-29. (canceled)

30. A method for treating metastatic cancer or cancer in an unresectable setting, wherein said method comprises administering, to a mammal identified as having a cancer comprising a reduced level of 5hmC, a high dose of AA in combination with one or more chemotherapeutic agents or one or more targeted therapies.

31. The method of claim 30, wherein said mammal is a human.

32. The method of claim 30, wherein said cancer is a kidney cancer.

33. The method of claim 30, wherein said cancer is a ccRCC.

34-39. (canceled)

40. A method for treating cancer in a localized setting, wherein said method comprises administering, to a mammal identified as having a cancer comprising a reduced level of 5hmC, a high dose of AA, one or more chemotherapeutic agents, or one or more targeted therapies.

41. The method of claim 40, wherein said mammal is a human.

42. The method of claim 40, wherein said cancer is a kidney cancer.

43. The method of claim 40, wherein said cancer is a ccRCC.

44-53. (canceled)
